(19) United States
(12) Patent Application Publication
(10) Pub. No.: US 2025/0345121 A1
(43) Pub. Date: Nov. 13, 2025

(54) METHODS AND APPARATUS FOR ASSESSING ANATOMICAL FIT OF AN INTRAVASCULAR BLOOD PUMP

(71) Applicant: Abiomed, Inc., Danvers, MA (US)

(72) Inventors: Christopher Sheils, Danvers, MA (US); James Melton, Danvers, MA (US)

(21) Appl. No.: 19/204,703

(22) Filed: May 12, 2025

Related U.S. Application Data
(60) Provisional application No. 63/646,257, filed on May 13, 2024.

(51) Int. Cl. A61B 34/10; A61B 34/00; G06T 7/00; G16H 10/60

(52) U.S. Cl. CPC A61B 34/10; A61B 34/25; G06T 7/0012; G16H 10/60; A61B 2034/104; A61B 2034/105; A61B 2034/108; G06T 2207/30048

(57) ABSTRACT

Methods and apparatus for assessing the fit of a blood pump are provided. The method includes receiving an anatomical model representing a cardiovascular system of a patient, determining, based on the anatomical model and one or more physical characteristics of a blood pump, a fit assessment of the blood pump within a portion of the cardiovascular system of the patient, and outputting an indication of the fit assessment on a user interface of a computing device.

### RELATED APPLICATIONS

This application claims the benefit under 35 U.S.C. § 119(e) to U.S. Provisional Patent Application No. 63/646,257, filed May 13, 2024, and titled, “METHODS AND APPARATUS FOR ASSESSING ANATOMICAL FIT OF AN INTRAVASCULAR BLOOD PUMP,” the entire contents of which is hereby incorporated by reference herein.

### TECHNICAL FIELD

This disclosure relates to techniques for assessing anatomical fit of a blood pump.

### BACKGROUND

Blood pump assemblies, such as intracardiac or intravascular blood pumps may be introduced in the heart to deliver blood from the heart into an artery. Such mechanical circulatory support devices are often introduced to support the function of the heart after a patient suffers a cardiac episode. One such class of devices is the set of devices known as the “Impella” heart pump, available from ABIOMED, Inc., Danvers, MA under the tradename Impella® heart pump. Some blood pump assemblies may be introduced percutaneously through the vascular system during a cardiac procedure. Specifically, blood pump assemblies can be inserted via a catheterization procedure through the femoral artery or the axillary/subclavian artery, into the ascending aorta, across the valve and into the left ventricle. The inserted blood pump assembly may be configured to pull blood from the left ventricle of the heart through a cannula and expels the blood into the aorta. A blood pump assembly may also be configured to pull blood from the inferior vena cava, superior vena cava, or atrium and to expel blood into the pulmonary artery. Some mechanical circulatory support devices are powered by an on-board motor, while others are powered by an external motor and a drive cable.

### SUMMARY

In some embodiments, a method is provided. The method includes receiving an anatomical model representing a cardiovascular system of a patient, determining, based on the anatomical model and one or more physical characteristics of a blood pump, a fit assessment of the blood pump within a portion of the cardiovascular system of the patient, and outputting an indication of the fit assessment on a user interface of a computing device.

In one aspect, the anatomical model is based, at least in part, on medical imaging information associated with the patient. In another aspect, the medical imaging information includes one or more computed tomography (CT) scans and/or one or more ultrasound scans of the patient. In another aspect, the method further includes receiving one or more measurements for one or more anatomical structures represented in the anatomical model, and determining the fit assessment is based on the anatomical model comprises determining the fit assessment based on the one or more measurements for the one or more anatomical structures. In another aspect, the one or more measurements for one or more anatomical structures represented in the anatomical model include one or more distances and/or diameters of the one or more anatomical structures. In another aspect, the one or more physical characteristics of the blood pump comprises one or more dimensions and/or shapes of the blood pump, and determining the fit assessment is further based on the one or more dimensions and/or shapes of the blood pump. In another aspect, the one or more measurements for one or more anatomical structures include one or more of: ventricle length, ventricle basal diameter, ventricle mid-cavity diameter, ventricle apex to base diameter, ascending aorta length, ascending aorta diameter, brachiocephalic artery diameter, subclavian artery diameter, axillary artery diameter, femoral artery diameter, iliac artery diameter, aortic arch radius, angle of left ventricle to aortic valve, inferior vena cava diameter, superior vena cava diameter, superior vena cava-right atrium junction diameter, right atrium diameter, tricuspid valve annulus diameter, pulmonary artery diameter, or pulmonary artery length.

In another aspect, the method further includes receiving a 3D model of the blood pump, the 3D model of the blood pump being generated based on the one or more physical characteristics of the blood pump, the anatomical model is a 3D anatomical model and determining the fit assessment of the blood pump is based on a comparison of the 3D model of the blood pump and the 3D anatomical model. In another aspect, determining the fit assessment of the blood pump based on a comparison of the 3D model of the blood pump and the 3D anatomical model comprises transforming the 3D model of the blood pump and the 3D anatomical model into a common coordinate space, and overlaying the 3D model of the blood pump and the 3D anatomical model to determine the fit assessment. In another aspect, the common coordinate space is a coordinate space of the 3D anatomical model.

In another aspect, the one or more physical characteristics of the blood pump include one or more of: a cannula length, a cannula diameter, an atraumatic tip length, a catheter length, a catheter diameter, a catheter bend, a pump housing length, a pump housing diameter, a motor housing length, a motor housing diameter, an inlet cage length, an inlet cage diameter, an outlet cage length, or an outlet cage diameter. In another aspect, determining a fit assessment of the blood pump within a portion of the cardiovascular system comprises determining whether an inlet and an outlet of the blood pump may be properly positioned across a valve of a heart of the patient. In another aspect, determining a fit assessment of the blood pump within a portion of the cardiovascular system comprises determining whether the blood pump can be delivered through a vasculature of the cardiovascular system of the patient during insertion of the blood pump into a heart of the patient. In another aspect, determining a fit assessment of the blood pump within a portion of the cardiovascular system comprises performing a simulation using the anatomical model and a model of the blood pump generated based on the one or more physical characteristics of the blood pump. In another aspect, the patient is a patient having a congenital heart abnormality. In another aspect, the patient is a pediatric patient. In another aspect, outputting an indication of the fit assessment comprises outputting a recommendation to use the blood pump and/or a recommendation to use a particular delivery technique to insert the blood pump. In another aspect, receiving an anatomical model representing a cardiovascular system of a patient comprises receiving, from an electronic health record associated with the patient, a set of medical images of the cardiovascular system of the patient, and generating the anatomical model based on the set of medical images.

In some embodiments, a method is provided. The method includes receiving medical imaging information for a patient, generating based on the medical imaging information, a 3D anatomical model representing a cardiovascular system of the patient, determining one or more measurements of one or more anatomical structures represented in the 3D anatomical model, receiving a 3D model of a blood pump, the 3D model of the blood pump being generated based on one or more physical characteristics of the blood pump, and performing a fit assessment of the blood pump within a portion of the cardiovascular system of the patient based on the 3D anatomical model and the 3D model of the blood pump.

In one aspect, the method further includes outputting an indication of the fit assessment on a user interface associated with a computing device. In another aspect, determining one or more measurements of one or more anatomical structures represented in the 3D anatomical model comprises determining one or more distances between two anatomical structures and/or diameters of the one or more anatomical structures. In another aspect, determining one or more measurements of one or more anatomical structures represented in the 3D anatomical model comprises determining a distance between two aspects of an anatomical structure. In another aspect, the one or more physical characteristics of the blood pump includes one or more dimensions and/or shapes of the blood pump. In another aspect, performing the fit assessment comprises performing a simulation using the 3D anatomical model and the 3D model of the blood pump. In another aspect, performing the fit assessment comprises comparing at least one of the one or more physical characteristics of the blood pump with at least one of the one or more measurements of one or more anatomical structures represented in the 3D anatomical model. In another aspect, determining a fit assessment comprises determining whether an inlet and an outlet of the blood pump may be properly positioned across a valve of a heart of the patient. In another aspect, determining a fit assessment comprises determining whether the blood pump can be delivered through a vasculature of the cardiovascular system of the patient during insertion of the blood pump into a heart of the patient. In another aspect, the patient is a patient having a congenital heart abnormality. In another aspect, the patient is a pediatric patient.

In some embodiments, a method is provided. The method includes receiving, via a user interface, a selection of a first blood pump to insert in a patient, performing a fit assessment of the first blood pump in a portion of a cardiovascular system of the patient based on a patient-specific anatomical model representing the cardiovascular system of the patient and a model of the first blood pump, and outputting a recommendation to treat the patient by inserting the first blood pump in the cardiovascular system of the patient when the fit assessment indicates that the first blood pump will fit within the portion of the cardiovascular system of the patient.

In one aspect, the method further includes treating the patient by inserting the first blood pump in the cardiovascular system of the patient after receiving the recommendation. In another aspect, the method further includes determining whether alternate options for blood pumps are available when the fit assessment indicates that the first blood pump will not fit within the portion of the cardiovascular system of the patient. In another aspect, the method further includes receiving a selection of a second blood pump when it is determined that alternate options for blood pumps are available, performing a fit assessment of the second blood pump in a portion of a cardiovascular system of the patient based on a patient-specific anatomical model representing the cardiovascular system of the patient and a model of the second blood pump, and outputting a recommendation to treat the patient by inserting the second blood pump in the cardiovascular system of the patient when the fit assessment indicates that the second blood pump will fit within the portion of the cardiovascular system of the patient. In another aspect, the first blood pump includes a first configurable portion, and receiving a selection of the second blood pump comprises receiving a user input to modify the first configurable portion of the first blood pump, wherein the second blood pump comprises the first blood pump having the first configurable portion modified based on the user input.

In another aspect, the method further includes outputting an indication that there are no suitable blood pumps when it is determined there are no alternate options for blood pumps available. In another aspect, the first blood pump includes at least one configurable portion, and receiving a selection of the first blood pump comprises receiving information associated with the at least one configurable portion of the first blood pump. In another aspect, receiving information associated with the at least one configurable portion comprises receiving length, shape, and/or material property information for the at least one configurable portion. In another aspect, the material property information includes a flexibility attribute of the at least one configurable portion. In another aspect, the method further includes displaying on the user interface, a first graphical representation of at least a portion of the patient-specific anatomical model and a second graphical representation of the first blood pump and displaying the second graphical representation of the first blood pump comprises indicating the at least one configurable portion of the first blood pump on the second graphical representation.

In another aspect, the method further includes displaying, on the user interface, a simulation tool, and performing a fit assessment of the first blood pump in a portion of a cardiovascular system of the patient comprises performing the fit assessment using the simulation tool. In another aspect, determining a fit assessment comprises determining whether an inlet and an outlet of the first blood pump may be properly positioned across a valve of a heart of the patient. In another aspect, determining a fit assessment comprises determining whether the first blood pump can be delivered through a vasculature of the cardiovascular system of the patient during insertion of the first blood pump into a heart of the patient. In another aspect, the patient is a patient having a congenital heart abnormality. In another aspect, the patient is a pediatric patient.

In some embodiments, a method of treatment is provided. The method includes determining, based on a 3D anatomical model representing a cardiovascular system of a patient and a 3D model of a blood pump, a fit assessment of the blood pump within a portion of the cardiovascular system of the patient, wherein the 3D model of the blood pump includes one or more physical characteristics of the blood pump, and treating the patient by delivering the blood pump through the cardiovascular system into a heart of the patient when the fit assessment indicates that the blood pump will fit within the portion of the cardiovascular system, operating the blood pump.

### BRIEF DESCRIPTION OF DRAWINGS

The foregoing and other objects and advantages will be apparent upon consideration of the following detailed description, taken in conjunction with the accompanying drawings, in which like reference characters refer to like parts throughout, and in which:

FIG. 1 is an illustration of a first example blood pump, according to an embodiment of the present disclosure.

FIG. 2 is an illustration of a second example blood pump, according to an embodiment of the present disclosure.

FIG. 3 is a flowchart of a process for determining a fit assessment of a blood pump, according to an embodiment of the present disclosure.

FIG. 4 is a flowchart of a process for generating and using a 3D model of a cardiovascular system of a patient to determine a fit of a blood pump, according to an embodiment of the present disclosure.

FIG. 5A schematically illustrates a process for generating a 3D model of a cardiovascular system of a patient based on medical imaging information for the patient, according to an embodiment of the present disclosure.

FIG. 5B schematically illustrates different measurements of patient anatomy that may be used to assess fit of a blood pump, according to an embodiment of the present disclosure.

FIG. 6 is a flowchart of a process for performing a simulation of delivery and/or placement of a blood pump in a patient based on an anatomical model and a blood pump model, according to an embodiment of the present disclosure.

FIG. 7 is a flowchart of a process for providing a treatment to a patient based on a fit assessment of a blood pump using on a patient-specific anatomical model and a blood pump model, according to an embodiment of the present disclosure.

### DETAILED DESCRIPTION

A cardiac support device (e.g., an intravascular blood pump), also referred to herein as a “blood pump,” a “heart pump,” or simply a “pump,” when properly positioned in a patient's heart may be controlled to pump blood from an inlet of the pump located on a first side of a valve in the patient's heart to an outlet of the pump located on a second side of the valve. In this way, the pump when in operation may at least partially replace or supplement the pumping action of the patient's native heart function. Prior to inserting the pump through the patient's vasculature and into the patient's heart, it may be challenging to determine whether the pump can be positioned correctly, especially for blood pumps that have a fixed size (e.g., blood pumps that are designed for use in adult patients with typical (e.g., average) cardiovascular system dimensions).

Some patients in need of cardiac support may have cardiovascular systems with anatomical dimensions that are different from the anatomical dimensions assumed when a blood pump was designed. For instance, patients with congenital heart abnormalities, pediatric patients, or petite patients may have smaller and/or non-standard anatomical dimensions compared with typical adult patients. The population of adults with congenital cardiac abnormalities is growing as pediatric survival with such conditions increases. Physicians have increasingly elected to use cardiac support devices with patients having congenital heart abnormalities or other patients with “non-standard” adult cardiovascular anatomies who are in need of mechanical circulatory support. Due to such patients having anatomical characteristics that differ from those assumed when a particular cardiac support device was designed, it may be challenging to determine whether the particular cardiac support device can be safely delivered through the patient's vasculature and/or positioned properly in a patient's heart to operate effectively. The inventors have recognized and appreciated that assessing the fit of a cardiac support device for a particular patient may be improved by using a 3D model of the patient's cardiovascular anatomy and information about the dimensions and/or shape of the cardiac support device to be used. In this way, selection of an appropriate cardiac support device may be tailored based on a patient's anatomy to better ensure that the device can be routed and positioned effectively once inserted.

Computed tomography (CT) scans, ultrasound scans, and/or other medical imaging techniques may be used to generate images that enable a physician to examine a patient's cardiovascular anatomy. In some embodiments, such medical imaging information may be used to generate a model (e.g., a 3D model) that represents the patient's cardiovascular anatomy. The model representing the patient's cardiovascular anatomy may be used to assess the fit of one or more mechanical circulatory support device options for the patient. For example, a physician may use the model representing the patient's cardiovascular anatomy to confirm that the patient's aortic arch is large enough to accommodate a particular blood pump and/or to ensure that the blood pump inlet and blood pump outlet could be properly positioned in the patient's heart. In some embodiments described in more detail herein, a fit of a particular blood pump may be assessed in view of a model representing a patient's cardiovascular anatomy by generating a model of the blood pump that can be compared to the cardiovascular anatomy model (e.g., using a simulation).

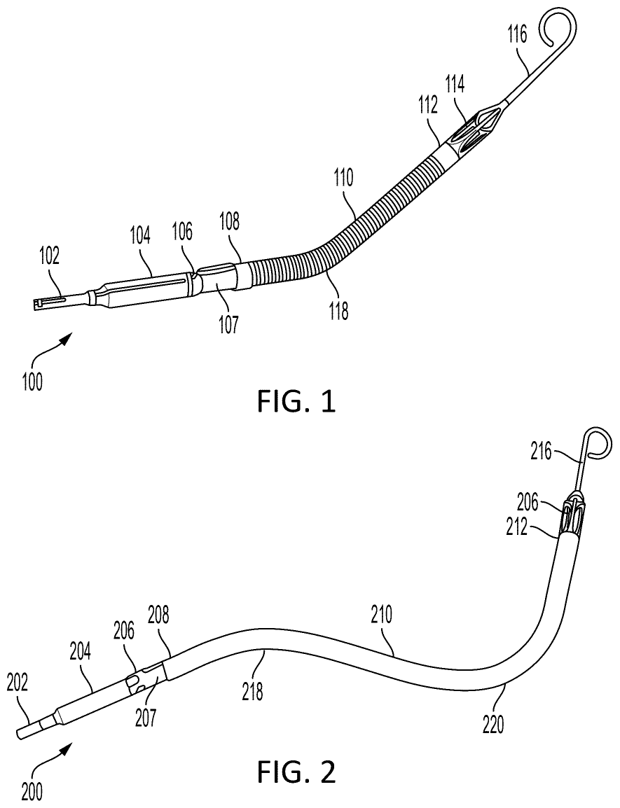

Turning to the figures, FIG. 1 shows an exemplary blood pump for use with some embodiments as disclosed herein. FIG. 1 depicts an exemplary intracardiac blood pump assembly 100 adapted for left heart support, in accordance with aspects of the disclosure. As shown in FIG. 1, an intracardiac blood pump assembly adapted for left heart support may include an elongate catheter 102, a motor 104, a cannula 110, a blood inflow cage 114 arranged at or near the distal end 112 of the cannula 110, a blood outflow cage 106 arranged at or near the proximal end 108 of the cannula 110, and an optional atraumatic extension 116 arranged at the distal end of the blood inflow cage 114.

In some aspects of the technology, motor 104 may be configured to rotatably drive an impeller (not shown), thereby generating suction sufficient to draw blood into cannula 110 through the blood inflow cage 114, and to expel the blood out of cannula 110 through the blood outflow cage 106. In that regard, the impeller may be positioned distal of the blood outflow cage 106, for example, within the proximal end 108 of the cannula 110 or within a pump housing 107 coupled to the proximal end 108 of the cannula 110. In some aspects of the technology, rather than the impeller being driven by an onboard motor 104, the impeller may instead be coupled to an elongate drive shaft (or drive cable) which is driven by a motor located external to the patient.

Catheter 102 may house electrical lines coupling the motor 104 to one or more electrical controllers and/or sensors. Alternatively, where the impeller is driven by an external motor, an elongate drive shaft may pass through catheter 102. Catheter 102 may also include a purge fluid conduit, a lumen configured to receive a guidewire, etc. Catheter 102 may include a preformed bend to accommodate certain patient anatomies. In some embodiments, the catheter 102 may bend inside of the patient's vasculature in response to the body temperature of the patient.

The blood inflow cage 114 may include one or more apertures or openings configured to allow blood to be drawn into cannula 110 when the motor 104 is operating. Likewise, blood outflow cage 106 may include one or more apertures or openings configured to allow blood to flow from the cannula 110 out of the intracardiac blood pump assembly 100. Blood inflow cage 114 and outflow cage 106 may be composed of any suitable bio-compatible material(s). For example, blood inflow cage 114 and/or blood outflow cage 106 may be formed out of bio-compatible metals such as stainless steel, titanium, or biocompatible polymers such as polyurethane. In addition, the surfaces of blood inflow cage 114 and/or blood outflow cage 106 may be treated in various ways, including, but not limited to etching, texturing, or coating or plating with another material. For example, the surfaces of blood inflow cage 114 and/or blood outflow cage 106 may be laser textured.

Cannula 110 may include a flexible hose portion. For example, cannula 110 may be composed, at least in part, of a polyurethane material. In addition, cannula 110 may include a shape-memory material. For example, cannula 110 may comprise a combination of a polyurethane material and one or more strands or coils of a shape-memory material such as Nitinol. Cannula 110 may be formed such that it includes one or more bends or curves in its relaxed state, or it may be configured to be straight in its relaxed state. In that regard, as shown in the exemplary arrangement of FIG. 1, the cannula 110 may have a single pre-formed anatomical bend 118 based on the portion of the left heart in which it is intended to operate. Despite this bend 118, the cannula 110 may nevertheless also be flexible, and may thus be capable of straightening (e.g., during insertion over a guidewire), or bending further (e.g., in a patient whose anatomy has tighter dimensions). Further in that regard, cannula 110 may include a shape-memory material configured to allow the cannula 110 to be a different shape (e.g., straight or mostly straight) at room temperatures, and to form bend 118 once the shape-memory material is exposed to the heat of a patient's body.

Atraumatic extension 116 may assist with stabilizing and positioning the intracardiac blood pump assembly 100 in the correct position in the patient's heart. Atraumatic extension 116 may be solid or tubular. If tubular, atraumatic extension 116 may be configured to allow a guidewire to be passed through it to further assist in the positioning of the intracardiac blood pump assembly 100. Atraumatic extension 116 may be any suitable size. For example, atraumatic extension 116 may have an outer diameter in the range of 4-8 Fr. Atraumatic extension 116 may be composed, at least in part, of a flexible material, and may be any suitable shape or configuration such as a straight configuration, a partially curved configuration, a pigtail-shaped configuration as shown in the example of FIG. 1, etc. Atraumatic extension 116 may also have sections with different stiffnesses. For example, atraumatic extension 116 may include a proximal section that is stiff enough to prevent it from buckling, thereby keeping the blood inflow cage 114 in the desired location, and a distal section that is softer and has a lower stiffness, thereby providing an atraumatic tip for contact with a wall of the patient's heart and to allow for guidewire loading. In such a case, the proximal and distal sections of the atraumatic extension 116 may be composed of different materials or may be composed of the same material with the proximal and distal sections being treated to provide different stiffnesses.

Notwithstanding the foregoing, as mentioned above, atraumatic extension 116 is an optional structure. In that regard, the present technology may also be used with intracardiac blood pump assemblies and other intracardiac devices that include extensions of different types, shapes, materials, and qualities. Likewise, the present technology may be used with intracardiac blood pump assemblies and other intracardiac devices that have no distal extensions of any kind.

As described herein, the intracardiac blood pump assembly 100 may be inserted percutaneously. For example, when used for left heart support, intracardiac blood pump assembly 100 may be inserted via a catheterization procedure through the femoral artery or axillary artery, into the aorta, across the aortic valve, and into the left ventricle. Once positioned in this way, the intracardiac blood pump assembly 100 may deliver blood from the blood inflow cage 114, which sits inside the left ventricle, through cannula 110, to the blood outflow cage 106, which sits inside the ascending aorta. In some aspects of the technology, intracardiac blood pump assembly 100 may be configured such that bend 118 will rest against a predetermined portion of the patient's heart when the intracardiac blood pump assembly 100 is in a desired location. Likewise, the atraumatic extension 116 may be configured such that it rests against a different predetermined portion of the patient's heart when the intracardiac blood pump assembly 100 is in the desired location.

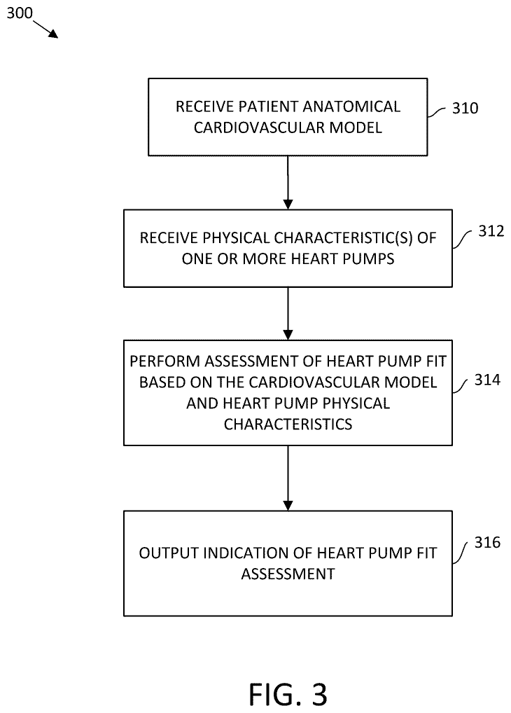

FIG. 2 depicts an exemplary intracardiac blood pump assembly 200 adapted for right heart support, in accordance with aspects of the disclosure. As shown in FIG. 2, an intracardiac blood pump assembly adapted for right heart support may include an elongate catheter 202, a motor 204, a cannula 210, a blood inflow cage 214 arranged at or near the proximal end 208 of the cannula 210, a blood outflow cage 206 arranged at or near the distal end 212 of the cannula 210, and an optional atraumatic extension 216 arranged at the distal end of the blood outflow cage 206.

As with the exemplary assembly of FIG. 1, motor 204 may be configured to rotatably drive an impeller (not shown), thereby generating suction sufficient to draw blood into cannula 210 through the blood inflow cage 214, and to expel the blood out of cannula 210 through the blood outflow cage 206. In that regard, the impeller may be positioned distal of the blood inflow cage 214, for example, within the proximal end 208 of the cannula 210 or within a pump housing 207 coupled to the proximal end 208 of the cannula 210. Here as well, in some aspects of the technology, rather than the impeller being driven by an onboard motor 204, the impeller may instead be coupled to an elongate drive shaft (or drive cable) which is driven by a motor located external to the patient.

The cannula 210 of FIG. 2 may serve the same purpose and/or may have the same properties and features described above with respect to cannula 110 of FIG. 1. However, as shown in the exemplary arrangement of FIG. 2, the cannula 210 may have two pre-formed anatomical bends 218 and 220 based on the portion of the right heart in which it is intended to operate. Here again, despite the existence of bends 218 and 220, the cannula 210 may nevertheless also be flexible, and may thus be capable of straightening (e.g., during insertion over a guidewire), or bending further (e.g., in a patient whose anatomy has tighter dimensions). Further in that regard, cannula 210 may include a shape-memory material configured to allow the cannula 210 to be a different shape (e.g., straight or mostly straight) at room temperatures, and to form bends 218 and/or 220 once the shape-memory material is exposed to the heat of a patient's body. Furthermore, in some embodiments, cannula 210 may also not include bends 218 and/or 220. In such embodiments, cannula 210 may be relatively straight during insertion and capable of bending to accommodate patients' anatomy once the pump is positioned in the patient.

The catheter 202 and atraumatic extension 216 of FIG. 2 may serve the same purpose and/or may have the same properties and features described above with respect to catheter 102 and atraumatic extension 116 of FIG. 1. Likewise, other than being located at opposite ends of the cannula from those of FIG. 1, the blood inflow cage 214 and blood outflow cage 206 of FIG. 2 may be similar to the blood inflow cage 114 and blood outflow cage 106 of FIG. 1, and thus may have the same properties and features described above. As will be appreciated, although shown with an atraumatic extension 216 in FIG. 2, the pump may not include an atraumatic extension.

Like the exemplary assembly of FIG. 1, the intracardiac blood pump assembly 200 of FIG. 2 may also be inserted percutaneously. For example, when used for right heart support, intracardiac blood pump assembly 200 may be inserted via a catheterization procedure through the femoral vein, into the inferior vena cava, through the right atrium, across the tricuspid valve, into the right ventricle, through the pulmonary valve, and into the pulmonary artery. Once positioned in this way, the intracardiac blood pump assembly 200 may deliver blood from the blood inflow cage 214, which sits inside the inferior vena cava, through cannula 210, to the blood outflow cage 206, which sits inside the pulmonary artery. In some embodiments, the intracardiac blood pump assembly 200 may be inserted via the internal jugular (IJ) vein. For example, in such embodiments, intracardiac blood pump assembly 200 may be inserted via a catheterization procedure through the IJ vein, into the superior vena cava, through the right atrium, across the pulmonary valve, and into the pulmonary artery. Once positioned in this way, the intracardiac blood pump assembly 200 may deliver blood from the blood inflow cage 214, which sits inside the superior vena cava, through cannula 210, to the blood outflow cage 206, which sits inside the pulmonary artery.

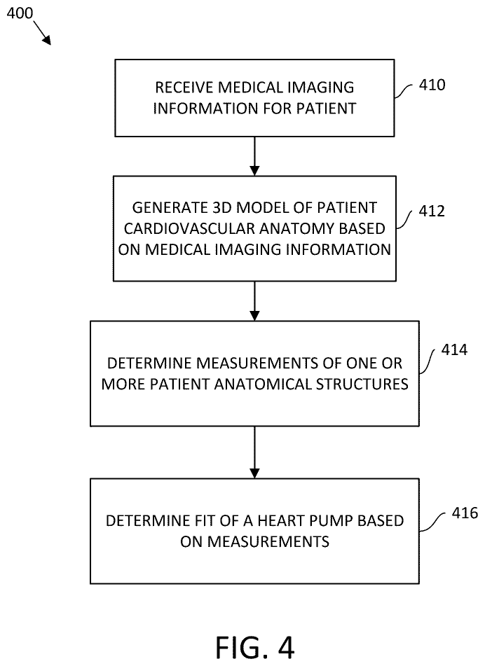

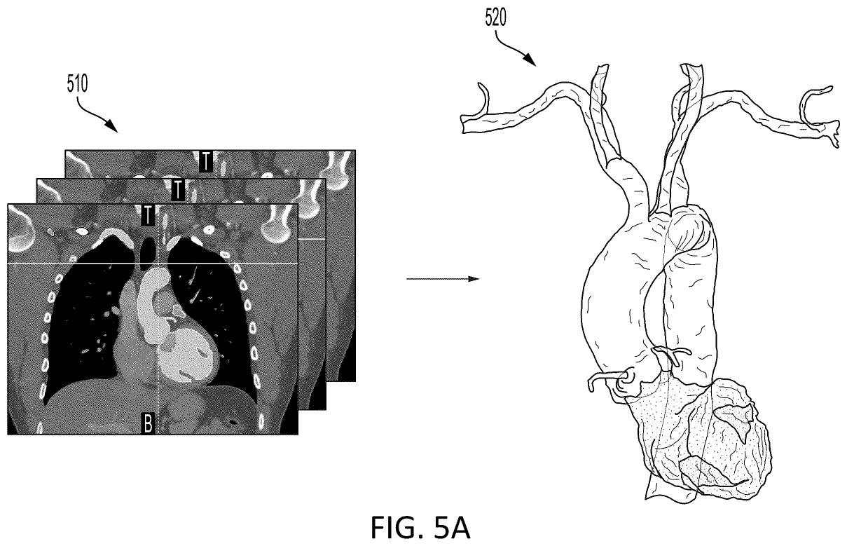

FIG. 3 shows a flowchart of a process 300 for assessing the fit of heart pump based on medical imaging information for a patient that represents at least a portion of the patient's cardiovascular system anatomy, in accordance with some embodiments of the present disclosure. Process 300 may begin in act 310, where a patient anatomical cardiovascular model is received. For instance, a 3D model representing the patient's heart and vasculature and may be generated based on medical imaging data and the 3D model may be provided to a computing system configured to perform process 300. An example of generating a patient anatomical cardiovascular model in accordance with some embodiments, is described in connection with the process shown in FIG. 4. Process 300 may then proceed to act 312, where one or more physical characteristics of one or more heart pumps may be received. For example, one or more dimensions (e.g., width, length) and/or shapes of the heart pump(s) may be received. In some embodiments, the physical characteristic(s) of the heart pump(s) received in act 312 may be embodied as a model (e.g., a 3D model) of the heart pump(s). In some embodiments, the physical characteristic(s) of the heart pump(s) received in act 312 may include one or more numerical values associated with all or a portion of the corresponding heart pump.

Process 300 may then proceed to act 314, where an assessment of heart pump fit may be performed based on the patient anatomical cardiovascular model and the physical characteristics for a particular heart pump. It should be appreciated that act 314 may be repeated for each of a plurality of heart pumps when multiple heart pumps are considered for a particular patient. In some embodiments, a user interface may be presented to a user (e.g., a physician) on a display of a computing device, and the user may select a particular heart pump to assess fit, and the corresponding physical characteristics for the selected heart pump may be used in act 314. The fit assessment may be performed in any suitable way. In some embodiments, the patient anatomical cardiovascular model received in act 310 may be a 3D model of the patient's vasculature, and the physical characteristic(s) of a heart pump received in act 312 may be represented in a 3D model of the pump. In such embodiments, the 3D model of the heart pump may be spatially overlaid on the 3D model representing the patient's cardiovascular anatomy to determine whether the pump can be properly and safely delivered and positioned in the patient's cardiovascular system. In some embodiments, the model may be dynamic such that the user may be able to simulate how the heart pump may react relative to particular portions of the patient's anatomy during delivery and/or positioning of the pump. In some embodiments, the physical characteristic(s) of the heart pump may include numerical values describing the dimensions and/or shape of the pump. In such embodiments, the fit assessment of the heart pump may be determined by first generating a 3D model of the heart pump based on the received numerical values and then comparing the 3D heart pump model to the cardiovascular model of the patient as described above. Additionally or alternatively, the fit assessment for the pump may be performed by determining measurements (e.g., lengths, diameters) of one or more anatomical structures using the patient's anatomical cardiovascular model and comparing the determined anatomical measurements to the numerical values representing the physical characteristics of the heart pump. Examples of measurements of one or more anatomical structures include, but are not limited to ventricle length, ventricle basal diameter, ventricle mid-cavity diameter, ventricle apex to base diameter, ascending aorta length, ascending aorta diameter, brachiocephalic artery diameter, subclavian artery diameter, axillary artery diameter, femoral artery diameter, iliac artery diameter, aortic arch radius, angle of left ventricle to aortic valve, inferior vena cava diameter, superior vena cava diameter, superior vena cava-right atrium junction diameter, right atrium diameter, tricuspid valve annulus diameter, pulmonary artery diameter, or pulmonary artery length.

Process 300 may then proceed to act 316, where an indication of the heart pump fit assessment may be output. The indication of heart pump fit assessment may be provided in any suitable way. For example, in some embodiments, a yes/no decision indicating whether a selected heart pump will fit within the anatomy of the patient may be provided. In some embodiments, the output indication may include a recommendation to use a particular heart pump and/or to use a particular delivery technique for inserting the particular heart pump in the patient. For instance, if the patient's cardiovascular anatomy includes a congenital abnormality (e.g. a narrow artery) that would prevent delivery of a heart pump into the patient's heart using a typical delivery technique (e.g., femoral artery insertion), but the pump would otherwise be able to be positioned properly in the patient's heart, a delivery technique other than the typical delivery technique for a particular heart pump model may be recommended. In some embodiments, a recommendation of another pump other than the selected heart pump (e.g. a heart pump with different dimensions and/or material characteristics) may be provided to the user.

FIG. 4 shows a flowchart of a process 400 for performing a fit assessment of a heart pump based on measurements of one or more patient anatomical structures, in accordance with some embodiments of the present disclosure. Process 400 may begin in act 410, where medical imaging information for a patient is received. In some embodiments, the medical imaging information may include a plurality of images from a computed tomography (CT) scan, an ultrasound scan, a positron emission tomography (PET) scan, or other suitable medical imaging scan. In some embodiments, the medical imaging information may be received directly from a medical imaging device configured to perform the medical imaging scan. In some embodiments, the medical imaging information may be received from an electronic health record (EHR) of a patient or may be received from any other suitable source. For example, the patient may need urgent cardiac support and time may not permit conducting an additional medical imaging scan. In such a situation, one or more medical imaging scans that have previously been completed may be used. In some instances, the patient may be stabilized using support techniques other than insertion of a cardiac support device (e.g., by pharmaceutical means) or using a different type of cardiac support device (e.g., another type of heart pump configured for short term use), and a medical imaging scan may be conducted once the patient is stable.

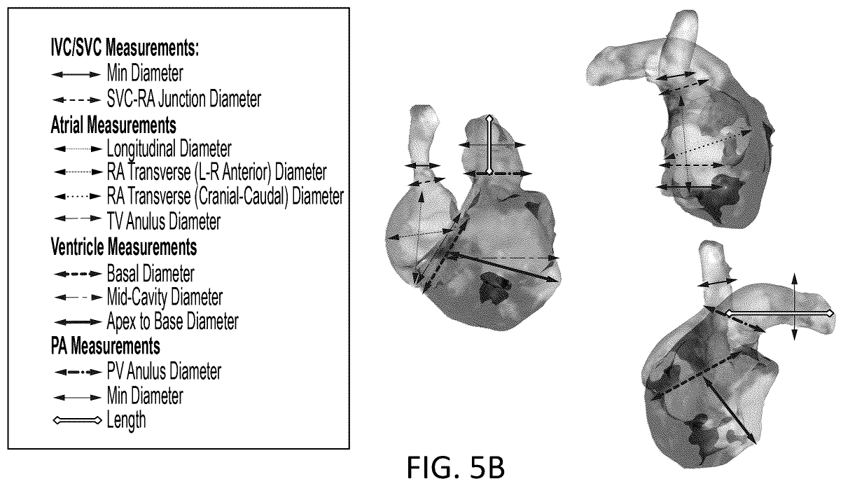

Process 400 may then proceed to act 412, where a 3D model of the patient's cardiovascular anatomy may be generated based on the medical imaging information received in act 410. For instance, 3D reconstruction of at least a portion of the patient's cardiovascular anatomy may be performed based on the medical imaging information. FIG. 5A schematically illustrates an example process for generating a 3D model representing a patient's cardiovascular anatomy. In the example shown in FIG. 5A, a plurality of CT images 510 (e.g., a plurality of sagittal CT image slices) are received and cardiovascular anatomical structures of interest (e.g., the left ventricle and arteries from aortic root through the left and right axillary arteries) may be segmented from the CT images. The segmented anatomical structures may be used to generate a 3D model 520 of the cardiovascular anatomy for the patient. The 3D model 520 may be assembled using any suitable techniques. For example, in some embodiments, 3D model 520 may be created using commercially available CT scan reconstruction software. FIG. 5B schematically illustrates a plurality of measurements that may be determined based on 3D model of the cardiovascular anatomy for a patient (e.g., measurements for the right side of a patient's heart). Such measurements may be used to assess a fit of a blood pump (e.g., a blood pump configured to provide right heart support) within the patient's anatomy.

Process 400 may then proceed to act 414, where measurements of one or more patient anatomical structures may be determined. For example, one or more distances, diameters, and/or curvatures of anatomical structures of interest may be determined using measurement tools available within the anatomical modeling software or using any other suitable measurement technique. In some embodiments, the minimum diameter (e.g., the minimum internal diameter) of an artery may be determined by defining a centerline of the artery and measuring a distance from the centerline to the interior surface of the artery wall. In some embodiments, the minimum internal diameter of one or more of the following anatomical structures may be determined: the right axillary artery, the left axillary artery, the innominate artery, the carotid artery, the femoral artery, the iliac artery, the superior vena cava (SVC) artery, inferior vena cava artery, the pulmonary artery, and/or the junction of the SVC and right atrium. In some embodiments, the diameter of a particular portion of an artery may be determined. For instance, the diameter of the ostium of the innominate artery and/or the diameter of the ostium of the lower left subclavian artery may be determined. In some embodiments, the diameter of the right atrium may be determined. For example, the longitudinal diameter of the right atrium may be determined. The traverse diameter of the right atrium as measured from cranial-caudal may be determined. The traverse diameter of the right atrium as measured from left to right may be determined. The tricuspid valve annulus diameter may also be determined. The pulmonary valve annulus may also be determined. In some embodiments, distances between aspects of the same or different anatomical structures may be determined. For example, the distance from the aortic valve to the ostium of the innominate artery (along the centerline and/or along the vertical length of the aorta), the distance from the aortic valve to the location where the centerline of the innominate artery branches, the distance from the center of the aortic valve to the apex of the left ventricle, or any other suitable distance measurement may be determined. In some embodiments, the aortic arch radius may be determined. In some embodiments, the angle of the left ventricle to the aortic valve may be determined. In some embodiments, the length of the pulmonary artery may be determined. In some embodiments, certain diameters of the right ventricle may be determined. For example, the basal diameter, the mid-cavity diameter, and/or the apex to base diameter of the right ventricle may be determined. It should be appreciated, that the measurements determined in act 414 may be determined based on the medical imaging information received in act 410 and/or the 3D model generated in act 412. FIGS. 5A and 5B show examples of various measurements of anatomical structures overlaid on the 3D model of the patient's cardiovascular anatomy.

Process 400 may then proceed to act 416, where a fit assessment of a heart pump may be performed based on the measurements for one or more patient anatomical structures determined in act 414. The fit assessment of the heart pump may be performed in any suitable way. In some embodiments, measurements (e.g., dimensions and/or shapes) associated with the heart pump may be compared with the measurements (e.g., lengths, distances, diameters, curvatures) of the one or more patient anatomical structures determined in act 414 to determine whether the heart pump may be fit within the patient's anatomy. In some embodiments, a 3D model of all or a portion of the heart pump may be generated, and the fit assessment may be based on a comparison of the 3D model of the heart pump and at least a portion of a 3D model of the patient's cardiovascular anatomy to determine whether the heart pump will fit within the patient's anatomy. In some embodiments, the fit assessment may be determined by transforming the 3D model of the heart pump and the 3D anatomical model into a common coordinate space (e.g., a coordinate space of the 3D anatomical model), and overlaying the 3D model of the heart pump and the 3D anatomical model. As described herein, some embodiments are directed to a simulation tool, which a user (e.g., a physician) may use to simulate placement of one or more heart pumps within the patient's cardiovascular anatomy to assess fit. For instance, a rendering of the 3D model representing the heart pump may be overlaid on a rendering of the 3D model of the patient's cardiovascular anatomy and a user may manipulate the rendering(s) by translating and/or rotating the rendering(s) to assess whether the heart pump will fit within the patient's anatomy.

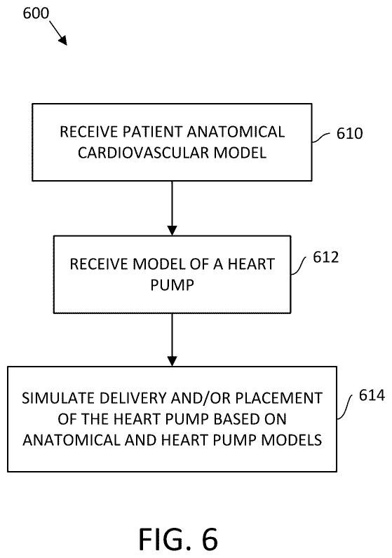

FIG. 6 shows a flowchart of a process 600 for assessing fit of a heart pump using a simulation tool, in accordance with some embodiments of the present disclosure. Process 600 may begin in act 610, where an anatomical cardiovascular model for a patient may be received. For example, a 3D anatomical cardiovascular model may be generated based on CT scan data for a patient as described in connection with process 400 shown in FIG. 4. Process 600 may then proceed to act 612, where a model describing physical characteristics (e.g., one or more dimensions, material properties, etc.) of a heart pump may be received. In some embodiments, the model of the heart pump may be a 3D model of the heart pump. In some embodiments, the model may a realistic representation of the heart pump. In some embodiments, the model may be a more abstract representation that represents the shape and size of the heart pump. In some embodiments, the model may include fewer than all components of the actual heart pump. Process 600 may then proceed to act 614, where the delivery and/or placement of the heart pump may be simulated based on the anatomical cardiovascular model and the heart pump model. For instance, some embodiments may provide a simulation tool configured to be provided in a graphical user interface displayed on a computing device. The graphical user interface may display a first graphical representation of the anatomical cardiovascular model and a second graphical representation of the heart pump model. A physician or other user may interact with the user interface (e.g., by providing one or more user inputs) to assess the fit of the heart pump by translating and/or rotating the second graphical representation relative to the first graphical representation to determine, for example, whether the heart pump will fit entirely within the patient's anatomy. In some embodiments, the graphical representation of the heart pump may include annotations describing one or more characteristics (e.g., material properties or attributes, curved vs. straight) of one or more portions of the pump. For instance, certain portions of the pump may be rigid or flexible to facilitate insertion of the pump through the vasculature of the patient. Such material properties may be illustrated on the graphical representation of the heart pump model (e.g., using cross-hatching, texture, color, or some other indication) displayed in the graphical user interface. In some embodiments, the graphical representations of one or both of the models may include reference points, which may be interactive/changeable by a user and may be useful in assessing the fit of a heart pump within a patient's cardiovascular anatomy. For instance, such reference points may be used to determine whether the patient would be at high risk of suction events if the inlet of the heart pump is placed too close to a vessel wall when the heart pump is in operation inside the vessel.

In some embodiments, one or more portions of a heart pump may be configurable and/or interchangeable, and the simulation tool may enable the user to select different interchangeable portions to customize a heart pump for a particular patient based on that patient's cardiovascular anatomy. For example, one or more of the following pump components may have different possible/configurable lengths, diameters, and/or material properties: cannula, atraumatic tip, catheter, pump housing, motor housing, inlet cage, and outlet cage. In some embodiments, only certain pump components (e.g., inlet cage, outlet cage, cannula) may be configurable/interchangeable, whereas other pump components may not be configurable. In some embodiments, the simulation tool may enable the user to simulate delivery and/or placement of a selected heart pump within a patient's anatomy prior to insertion of the heart pump. In some embodiments, the simulation tool may recommend a delivery method (e.g., femoral insertion, via the internal jugular vein, axillary artery, etc.) based on a patient's anatomy.

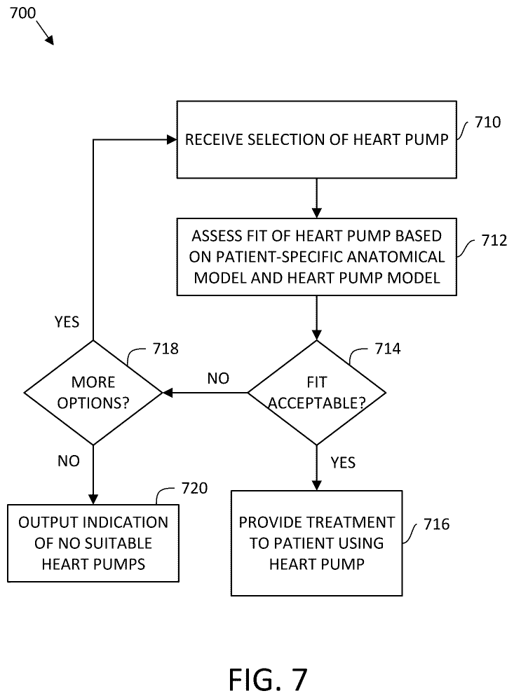

FIG. 7 shows a process 700 for treating a patient using a selected heart pump, in accordance with some embodiments of the present disclosure. Process 700 may begin in act 710, where a selection of a heart pump (e.g., Impella® CP, Impella® 5.5, Impella® RP, etc. available from ABIOMED, Inc.) is received. For instance, the selection of the heart pump may be received from a user interacting with a graphical user interface that presents the available options. In some embodiments, a listing of available heart pumps may be displayed on a graphical user interface (e.g., in a drop-down menu), and the user may select one of the available heart pumps. In some embodiments, the listing of available heart pumps may be filtered based on a particular FDA-approved indications for the pump and information about the patient (e.g., whether an intended use of the pump is ≤6 hours, ≤4 days, ≤14 days, etc., whether the patient is undergoing a high-risk percutaneous coronary intervention, whether the patient is a cardiogenic shock patient, whether the patient is a pediatric patient, etc.).

Process 700 may then proceed to act 712, where the fit of the heart up may be assessed based on a patient-specific anatomical model and a model of the selected heart pump, examples of which are described herein. Process 700 may then proceed to act 714, where it is determined whether the fit of the heart pump is acceptable based on the assessment. If it is determined that the fit is acceptable, process 700 may proceed to act 716, where the patient is provided treatment by inserting the selected heart pump. In embodiments in which one or more components of the heart pump are configurable/interchangeable, treating the patient in act 716 may include assembling the set of selected configurable/interchangeable components that have an acceptable fit prior to inserting the assembled “customized” or “personalized” heart pump into the patient's heart.

If it is determined in act 714 that the fit of the selected heart pump is not acceptable, process 700 may proceed to act 718 where it may be determined whether there are additional heart pump options available. If there are no other options available, process 700 may proceed to act 720 where an indication may be provided that there is no suitable heart pump to use with the particular patient. If it is determined in act 718 that there are additional heart pump options available, process 700 may return to act 710, where a new heart pump may be selected. In embodiments in which one or more components of the heart pump are configurable/interchangeable as described herein, selecting a new heart pump in act 710 may involve selecting one or more different configurable/interchangeable components of the previously selected heart pump rather than necessarily selecting a different model of heart pump. In some embodiments, the computing device associated with the graphical user interface may provide a recommendation for a heart pump to be used with a particular patient, where the recommendation is based, at least in part, on the anatomical cardiovascular model of the patient. A user may take such a recommendation into consideration when selecting a heart pump in act 710. Acts 710-718 may be repeated until a heart pump is determined to have an acceptable fit in act 714 or when it is determined in at 718 that there are no more heart pump options available.

In some embodiments, the techniques described herein may be used during a pre-planning stage (e.g., before insertion of the heart pump into the patient). In some embodiments, the techniques described herein may be used during a transition from providing support from one type of heart pump to another type of heart pump. For instance, the patient may be stabilized with a first type of heart pump (e.g., an Impella® CP pump) and it may be determined that the patient would benefit from additional flow. The techniques described herein may be used to determine whether a second type of heart pump capable of producing more flow (e.g., an Impella® 5.5 pump) would fit properly in the patient's anatomy if used to replace the first type of heart pump. In some embodiments, the techniques described herein may be used during insertion of a heart pump. For instance, the modeling techniques described herein may be used during insertion of the heart pump to facilitate positioning by providing a reference to a physician during the insertion procedure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including”, “comprising”, or “having”, “containing”, “involving”, and variations thereof herein, is meant to encompass the items listed thereafter and equivalents thereof as well as additional items.

In the claims, as well as in the specification above, all transitional phrases such as “comprising,” “including”, “carrying”, “having”, “containing”, “involving”, “holding”, “composed of”, and the like are to be understood to be open-ended, i.e., to mean including but not limited to. Only the transitional phrases “consisting of” and “consisting essentially of” shall be closed or semi-closed transitional phrases, respectively.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

1. A method, comprising:
receiving an anatomical model representing a cardiovascular system of a patient;
determining, based on the anatomical model and one or more physical characteristics of a blood pump, a fit assessment of the blood pump within a portion of the cardiovascular system of the patient; and
outputting an indication of the fit assessment on a user interface of a computing device.

2. The method of claim 1, wherein the anatomical model is based, at least in part, on medical imaging information associated with the patient.

3. (canceled)

4. The method of claim 1, further comprising:
receiving one or more measurements for one or more anatomical structures represented in the anatomical model,
wherein determining the fit assessment based on the anatomical model comprises determining the fit assessment based on the one or more measurements for the one or more anatomical structures.

5. The method of claim 4, wherein the one or more measurements for one or more anatomical structures represented in the anatomical model include one or more distances and/or diameters of the one or more anatomical structures.

6. The method of claim 4, wherein
the one or more physical characteristics of the blood pump comprises one or more dimensions and/or shapes of the blood pump, and
determining the fit assessment is further based on the one or more dimensions and/or shapes of the blood pump.

7. The method of claim 4, wherein the one or more measurements for one or more anatomical structures include one or more of: ventricle length, ventricle basal diameter, ventricle mid-cavity diameter, ventricle apex to base diameter, ascending aorta length, ascending aorta diameter, brachiocephalic artery diameter, subclavian artery diameter, axillary artery diameter, femoral artery diameter, iliac artery diameter, aortic arch radius, angle of left ventricle to aortic valve, inferior vena cava diameter, superior vena cava diameter, superior vena cava-right atrium junction diameter, right atrium diameter, tricuspid valve annulus diameter, pulmonary artery diameter, or pulmonary artery length.

8. The method of claim 1, further comprising:
receiving a 3D model of the blood pump, the 3D model of the blood pump being generated based on the one or more physical characteristics of the blood pump,
wherein the anatomical model is a 3D anatomical model and determining the fit assessment of the blood pump is based on a comparison of the 3D model of the blood pump and the 3D anatomical model.

9. The method of claim 8, wherein determining the fit assessment of the blood pump based on a comparison of the 3D model of the blood pump and the 3D anatomical model comprises:
transforming the 3D model of the blood pump and the 3D anatomical model into a common coordinate space; and
overlaying the 3D model of the blood pump and the 3D anatomical model to determine the fit assessment.

10. (canceled)

11. The method of claim 1, wherein the one or more physical characteristics of the blood pump include one or more of: a cannula length, a cannula diameter, an atraumatic tip length, a catheter length, a catheter diameter, a catheter bend, a pump housing length, a pump housing diameter, a motor housing length, a motor housing diameter, an inlet cage length, an inlet cage diameter, an outlet cage length, or an outlet cage diameter.

12. The method of claim 1, wherein determining a fit assessment of the blood pump within a portion of the cardiovascular system comprises determining whether an inlet and an outlet of the blood pump may be properly positioned across a valve of a heart of the patient.

13. The method of claim 1, wherein determining a fit assessment of the blood pump within a portion of the cardiovascular system comprises determining whether the blood pump can be delivered through a vasculature of the cardiovascular system of the patient during insertion of the blood pump into a heart of the patient.

14. The method of claim 1, wherein determining a fit assessment of the blood pump within a portion of the cardiovascular system comprises performing a simulation using the anatomical model and a model of the blood pump generated based on the one or more physical characteristics of the blood pump.

15-16. (canceled)

17. The method of claim 1, wherein outputting an indication of the fit assessment comprises outputting a recommendation to use the blood pump and/or a recommendation to use a particular delivery technique to insert the blood pump.

18. The method of claim 1, wherein receiving an anatomical model representing a cardiovascular system of a patient comprises:
receiving, from an electronic health record associated with the patient, a set of medical images of the cardiovascular system of the patient; and
generating the anatomical model based on the set of medical images.

19-29. (canceled)

30. A method, comprising:
receiving, via a user interface, a selection of a first blood pump to insert in a patient;
performing a fit assessment of the first blood pump in a portion of a cardiovascular system of the patient based on a patient-specific anatomical model representing the cardiovascular system of the patient and a model of the first blood pump; and
outputting a recommendation to treat the patient by inserting the first blood pump in the cardiovascular system of the patient when the fit assessment indicates that the first blood pump will fit within the portion of the cardiovascular system of the patient.

31. The method of claim 30, further comprising;
treating the patient by inserting the first blood pump in the cardiovascular system of the patient after receiving the recommendation.

32. The method of claim 31, further comprising:
determining whether alternate options for blood pumps are available when the fit assessment indicates that the first blood pump will not fit within the portion of the cardiovascular system of the patient;
receiving a selection of a second blood pump when it is determined that alternate options for blood pumps are available;
performing a fit assessment of the second blood pump in a portion of a cardiovascular system of the patient based on a patient-specific anatomical model representing the cardiovascular system of the patient and a model of the second blood pump; and
outputting a recommendation to treat the patient by inserting the second blood pump in the cardiovascular system of the patient when the fit assessment indicates that the second blood pump will fit within the portion of the cardiovascular system of the patient.

33. (canceled)

34. The method of claim 32, wherein
the first blood pump includes a first configurable portion, and
receiving a selection of the second blood pump comprises receiving a user input to modify the first configurable portion of the first blood pump, wherein the second blood pump comprises the first blood pump having the first configurable portion modified based on the user input.

35. (canceled)

36. The method of claim 30, wherein
the first blood pump includes at least one configurable portion, and
receiving a selection of the first blood pump comprises receiving information associated with the at least one configurable portion of the first blood pump.

37-38. (canceled)

39. The method of claim 36, further comprising:
displaying on the user interface, a first graphical representation of at least a portion of the patient-specific anatomical model and a second graphical representation of the first blood pump,
wherein displaying the second graphical representation of the first blood pump comprises indicating the at least one configurable portion of the first blood pump on the second graphical representation.

40-44. (canceled)
